(19) United States
(12) Patent Application Publication
(10) Pub. No.: US 2011/0056500 A1
(43) Pub. Date: Mar. 10, 2011

(54) BALLOON CATHETER FOR RESPIRATORY TRACT

(76) Inventors: Kyong-Min Shin, Seoul (KR); Yong-Hyun Won, Incheon-si (KR)

(21) Appl. No.: 12/875,709

(22) Filed: Sep. 3, 2010

(30) Foreign Application Priority Data
Sep. 7, 2009 (KR) 10-2009-0084126

(51) Int. Cl. A61M 29/02; A61M 16/00

(52) U.S. Cl. 128/207.14; 606/196

(57) ABSTRACT

Disclosed herein is a balloon catheter for the respiratory tract, which serves to widen the lumen of the respiratory tract when stricture or stenosis of the lumen occurs and is configured to enable a patient to breathe even during catheterization. The catheter includes a tube unit having a double structure consisting of an inner tube and an outer tube spaced apart from the inner tube to surround the inner tube, and a cylindrical balloon integrally connected to facing distal ends of the inner and outer tubes. The balloon is inflated to have a hollow cylindrical shape as inflation gas is supplied into the outer tube, thereby acting to widen the stenosed or narrowed lumen of the respiratory tract, and simultaneously, oxygen is supplied into the inner tube to pass through the cylindrical balloon, thereby enabling a patient to breathe and resulting in stability in catheterization.

### BACKGROUND OF THE INVENTION

1. Field of the Invention

The present invention relates to a balloon catheter for the respiratory tract, and more particularly, to a balloon catheter for the respiratory tract, which serves to widen the lumen of the respiratory tract when stricture or stenosis of the lumen occurs due to a lesion, and also, which is configured so as not to block the respiratory tract, thus enabling a patient to breathe even during catheterization.

2. Description of the Related Art

In general, lumen stenosis diseases, for example, stenosing airway diseases may be broadly classified, according to the anatomical site of a lesion, into intraluminal obstruction, extrinsic compression, and malacia.

Catheterization has been performed to remedy life-threatening dyspnea that is caused by extrinsic compression and malacia of the above mentioned stenosing airway diseases.

In catheterization, a catheter in a non-inflated state is inserted into the lumen of the respiratory tract until the catheter reaches the stenosed site of the lumen. Thereafter, the catheter is inflated to push the stenosed site outward, thereby serving to widen the lumen of the respiratory tract.

However, the catheter, which is inflated to have a balloon shape, may make it impossible for a patient to breathe while the catheter widens the stenosed respiratory tract.

In consideration of breathing of a patient, catheterization should be performed within an extremely limited time and thus, suffers from remarkable deterioration in stability.

### SUMMARY OF THE INVENTION

Therefore, the present invention has been made in view of the above problems, and it is one object of the present invention to provide a balloon catheter for the respiratory tract, in which a balloon is inserted into the lumen of the respiratory tract and then, is inflated to have a cylindrical shape as inflation gas is injected into the balloon, thereby serving to widen the lumen of the respiratory tract stenosed by a lesion.

It is another object of the present invention to provide a balloon catheter for the respiratory tract, in which a balloon is inflated to have a hollow cylindrical shape, thereby enabling a patient to breathe while widening the stenosed site of the respiratory tract.

In accordance with an aspect of the present invention, the above and other objects can be accomplished by the provision of a balloon catheter for a respiratory tract, which serves to widen the lumen of the respiratory tract stenosed by a lesion formed in the respiratory tract, the catheter including a tube unit having a double structure consisting of an inner tube and an outer tube spaced apart from the inner tube to surround the inner tube, and a cylindrical balloon integrally connected to facing distal ends of the inner and outer tubes, wherein the cylindrical balloon is inflated as inflation gas is supplied into the outer tube, thereby acting to widen the stenosed or narrowed lumen of the respiratory tract, and simultaneously, oxygen is supplied into the inner tube to pass through the cylindrical balloon to enable a patient to breathe.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a perspective view of a balloon catheter for the respiratory tract in accordance with one embodiment of the present invention;

FIG. 2 is a longitudinal sectional view of the portion A of FIG. 1;

FIG. 3 is a longitudinal sectional view of the portion B of FIG. 1;

FIG. 4 is a perspective view of a balloon catheter for the respiratory tract in accordance with another embodiment of the present invention;

FIG. 5 is a partial sectional perspective view of the portion C of FIG. 4;

FIG. 6 is a longitudinal sectional view of the portion D of FIG. 4;

FIG. 7 is an exemplary view illustrating implantation of the balloon catheter for the respiratory tract of FIG. 1; and

FIG. 8 is an exemplary view illustrating implantation of the balloon catheter for the respiratory tract of FIG. 4.

### DETAILED DESCRIPTION OF THE INVENTION

Hereinafter, the preferred embodiments of the present invention will be described in detail with reference to the accompanying drawings.

A balloon catheter for the respiratory tract in accordance with the present invention serves to widen the lumen of the respiratory tract 300 that is stenosed by a lesion formed in the respiratory tract 300. The balloon catheter is configured to enable a patient to breathe during catheterization to widen the lumen of the respiratory tract 300.

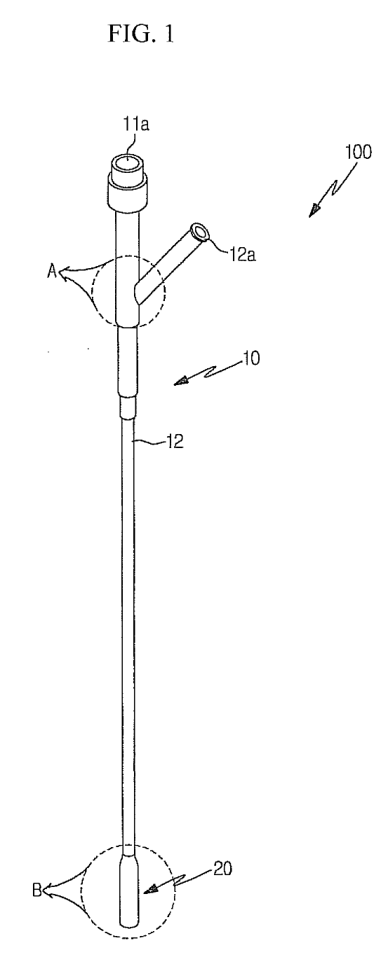

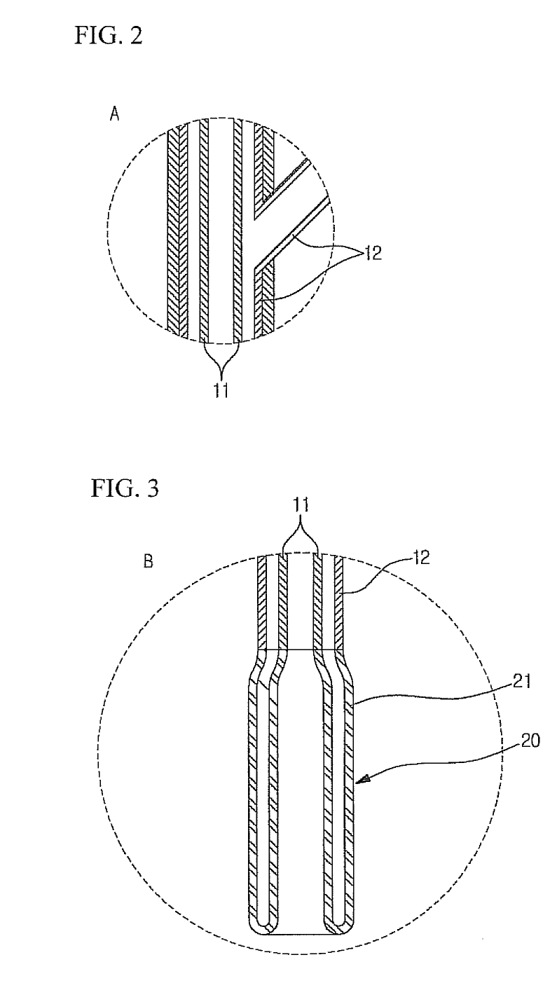

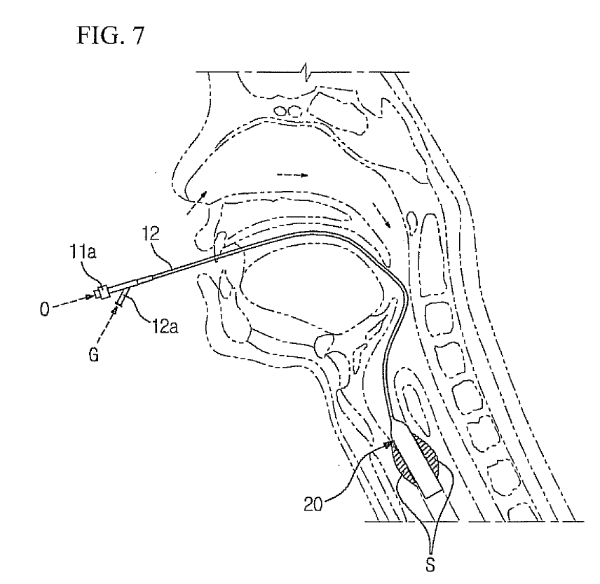

As illustrated in FIGS. 1 to 3 and 7, the balloon catheter 100 for the respiratory tract in accordance with one embodiment of the present invention includes a tube unit 10 and a cylindrical balloon 20.

The tube unit 10 has a double structure consisting of an inner tube 11 and an outer tube 12 spaced apart from the inner tube 11 to surround the inner tube 11.

In this case, both the inner tube 11 and the outer tube 12 are inserted into the respiratory tract 300. The inner tube 11 is provided at one end thereof with a connector lie such that the connector 11a is connected to a device (not shown) that supplies oxygen O into the inner tube 11. The outer tube 12 is provided at an outer peripheral surface thereof with a branched connector 12a such that the connector 12a is connected to a device (not shown) that supplies inflation gas G into a space between the inner tube 11 and the outer tube 12.

The cylindrical balloon 20 is integrally connected to facing distal ends of the inner and outer tubes 11 and 12.

Here, the cylindrical balloon 20 includes a single body 21, which is formed by overlapping synthetic vinyl in two layers and thus, has a bilinear U-shaped cross section.

In the single body 21 formed of the two synthetic vinyl layers, an upper end of the inner layer is connected to the inner tube 11 and an upper end of the outer layer is connected to the outer tube 12.

That is, the cylindrical balloon 20 may be made of synthetic vinyl or synthetic rubber that is easily expandable or contractable, and an upper end of the cylindrical balloon 20 may have a hollow donut shape when viewed in plan.

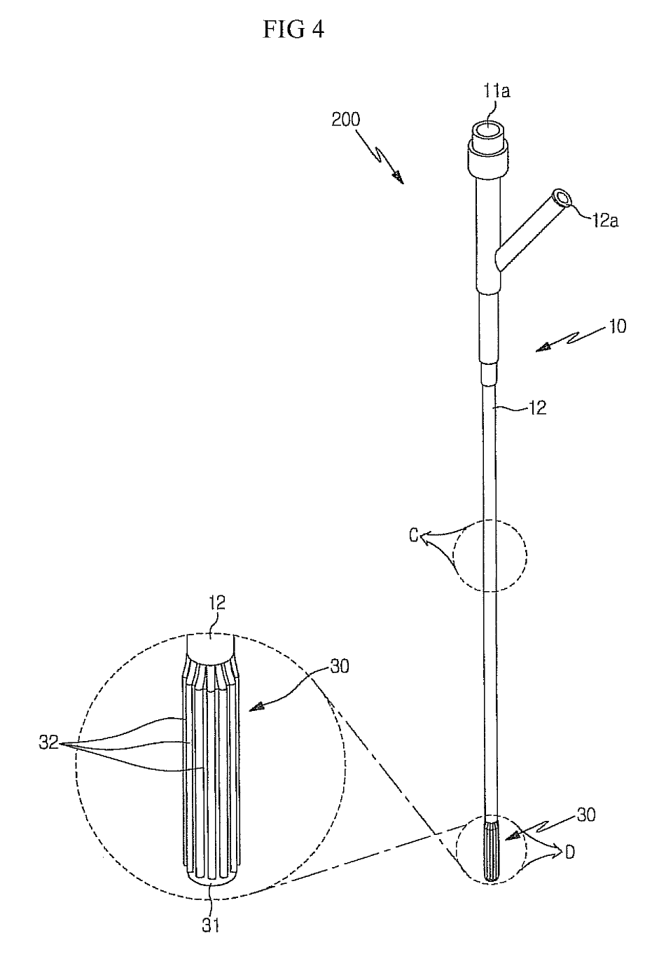

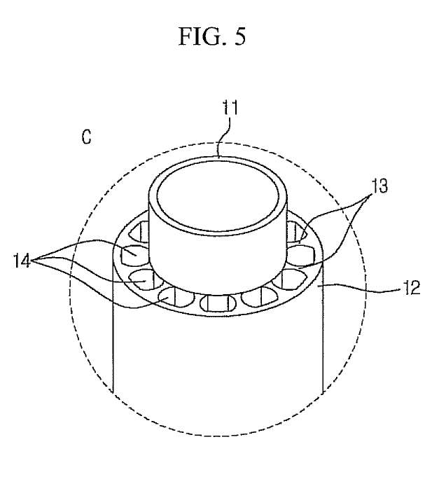

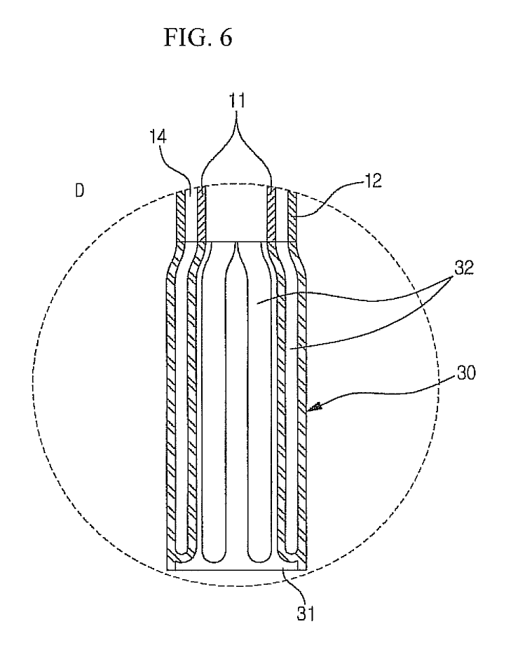

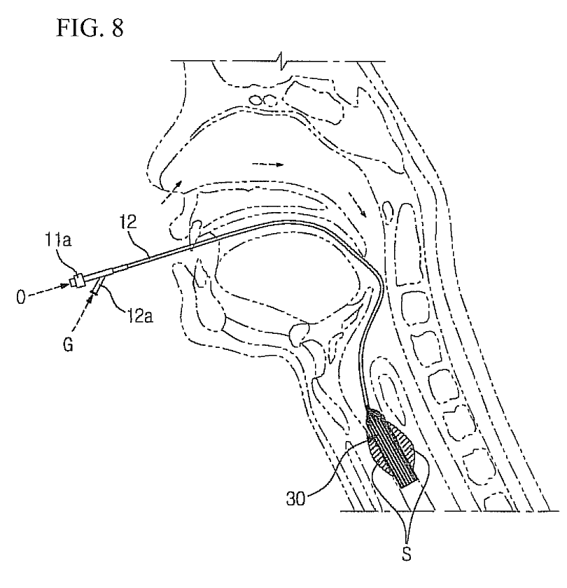

As illustrated in FIGS. 4 to 6 and 8, a balloon catheter 200 for the respiratory tract in accordance with another embodiment of the present invention includes the tube unit 10 and a cylindrical balloon 30.

The tube unit 10 has a double structure consisting of the inner tube 11 and the outer tube 12 spaced apart from the outer tube 12. In the present embodiment, the tube unit 10 further includes a plurality of guides 13, which are circumferentially arranged at a constant interval in the space between the inner tube 11 and the outer tube 12 and are configured to longitudinally connect the inner tube 11 and the outer tube 12 to each other, and a plurality of passages 14 defined between the respective neighboring guides 13.

In this case, the tube unit 10 may be formed by an injection molding method. The guides 13 serve to integrally connect the inner and outer tubes 11 and 12 to each other to reinforce the tube unit 10 and also, serve to define the passages 14 for movement of inflation gas G.

The cylindrical balloon 30 includes a single body 31, which is formed by overlapping synthetic vinyl in two layers and thus, has a bilinear U-shaped cross section.

In the single body 31 formed of the two synthetic vinyl layers, an upper end of the inner layer is connected to the inner tube 11 and an upper end of the outer layer is connected to the outer tube 12.

The cylindrical balloon 30 further includes a plurality of inflatable portions 32, which extend in a longitudinal direction of the cylindrical balloon 30 and are circumferentially arranged at a constant interval on the periphery of the single body 31 so as to be connected to the respective passages 14 of the tube unit 10.

In this case, even if any one of the inflatable portions 32 is damaged and fails to inflate, the balloon 30 is able to maintain a cylindrical shape thereof.

Hereinafter, the implantation process and operational effects of the catheter 100 or 200 in accordance with the present invention will be described.

Referring to FIGS. 1 to 3 and 7, the catheter 100 is inserted into the respiratory tract 300, having a stenosed site S due to a lesion, through the patient's mouth. In this case, the catheter 100 is inserted in a state in which inflation gas G is not yet injected into the cylindrical balloon 20, and is connected to the devices (not shown) for injection of oxygen O and inflation gas G prior to being inserted into the respiratory tract 300.

After the cylindrical balloon 20 of the catheter 100 is completely inserted into the stenosed site S, inflation gas G is injected into the outer tube 12 and simultaneously, oxygen O is injected into the inner tube 11.

As the injected inflation gas G moves in the space between the inner tube 11 and the outer tube 12 to thereby be filled in the cylindrical balloon 20, the single body 21 is inflated to have a cylindrical tube shape and thus, acts to push the inner wall surface of the stenosed site S of the respiratory tract 300 in all directions, thereby widening the stenosed site S.

After injection of the inflation gas G is completed, an entrance of the connector 12a provided at the outer tube is closed to prevent a reduction in the volume of the cylindrical balloon 20.

That is, the cylindrical balloon 20 is inflated as the inflation gas G is supplied into the outer tube 12, thereby acting to widen the stenosed or narrowed lumen of the respiratory tract 300, and simultaneously, the oxygen O is supplied through the inner tube 11 to pass through the cylindrical balloon 20, thereby enabling the patient to breathe.

Referring to FIGS. 4 to 6 and 8, the catheter 200 is inserted into the respiratory tract 300, having a stenosed site S due to a lesion, through the patient's mouth. In this case, the catheter 200 is inserted in a state in which inflation gas G is not yet injected into the cylindrical balloon 30, and is connected to the devices (not shown) for injection of oxygen O and inflation gas G prior to being inserted into the respiratory tract 300.

After the cylindrical balloon 30 of the catheter 100 is completely inserted into the stenosed site S, inflation gas G is injected into the outer tube 12 and simultaneously, oxygen O is injected into the inner tube 11.

As the injected inflation gas G moves into the respective inflatable portions 32 through the respective passages 14 of the tube unit 10, the cylindrical body 31 is inflated to have a cylindrical tube shape and thus, acts to push the inner wall surface of the stenosed site S of the respiratory tract 300 in all directions, thereby widening the stenosed site S.

After injection of the inflation gas G is completed, the entrance of the connector 12a provided at the outer tube is closed to prevent a reduction in the volume of the cylindrical balloon 30.

That is, the cylindrical balloon 30 is inflated as the inflation gas G is supplied into the outer tube 12, thereby acting to widen the stenosed or narrowed lumen of the respiratory tract 300, and simultaneously, the oxygen O supplied through the inner tube 11 passes through the cylindrical balloon 30 to enable the patient to breathe.

As described above, when a conventional balloon catheter is implanted into the lumen of the respiratory tract to widen the lumen stenosed by a lesion, the conventional catheter disadvantageously closes the lumen of the respiratory tract, thus making it impossible for the patient to breathe. Therefore, the conventional balloon catheter has an extremely limited time to widen the stenoise site of the lumen and thus, it may be necessary to perform catheterization several times. On the other hand, the catheter 100 or 200 of the present invention adopts a hollow cylindrical balloon suitable to open the respiratory tract 300 of the patient, thus enabling the patient to continue breathing and achieving an extended implantation time.

As is apparent from the above description, in a balloon catheter for the respiratory tract in accordance with the present invention, a balloon is inserted into the lumen of the respiratory tract and is inflated to have a cylindrical shape as inflation gas G is injected into the balloon, thereby effectively widening the lumen of the respiratory tract stenosed by a lesion.

In addition, as the balloon is inflated to have a hollow cylindrical shape, the balloon catheter has the effect of enabling a patient to breathe while widening the stenosed site of the lumen. This results in stability in catheterization.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

What is claimed is:

1. A balloon catheter for a respiratory tract, which serves to widen the lumen of the respiratory tract stenosed by a lesion formed in the respiratory tract, the catheter comprising:
a tube unit having a double structure consisting of an inner tube and an outer tube spaced apart from the inner tube to surround the inner tube; and
a cylindrical balloon integrally connected to facing distal ends of the inner and outer tubes,
wherein the cylindrical balloon is inflated as inflation gas is supplied into the outer tube, thereby acting to widen the stenosed or narrowed lumen of the respiratory tract, and simultaneously, oxygen is supplied into the inner tube to pass through the cylindrical balloon, thereby enabling a patient to breathe.

2. The catheter according to claim 1, wherein the cylindrical balloon includes a single body formed by overlapping synthetic vinyl in two layers to have a bilinear U-shaped cross section, and upper ends of the inner and outer layers of the single body are connected respectively to the distal ends of the inner and outer tubes.

3. A balloon catheter for a respiratory tract, which serves to widen the lumen of the respiratory tract stenosed by a lesion formed in the respiratory tract, the catheter comprising:
a tube unit having a double structure including inner and outer tubes spaced apart from each other, a plurality of guides circumferentially arranged at an interval in a space between the inner tube and the outer tube and configured to longitudinally connect the inner and outer tubes to each other, and a plurality of passages defined between the respective neighboring guides; and
a cylindrical balloon integrally connected to facing distal ends of the inner and outer tubes,
wherein the cylindrical balloon is inflated as inflation gas is supplied into the outer tube through the passages, thereby acting to widen the stenosed or narrowed lumen of the respiratory tract, and simultaneously, oxygen is supplied into the inner tube to pass through the cylindrical balloon, thereby enabling a patient to breathe.

4. The catheter according to claim 3, wherein the cylindrical balloon includes a single body formed by overlapping synthetic vinyl in two layers to have a bilinear U-shaped cross section, and a plurality of inflatable portions extending in a longitudinal direction of the cylindrical balloon are circumferentially arranged at an interval on the periphery of the single body so as to be connected to the respective passages of the tube unit.
